(19) United States
(12) Patent Application Publication
(10) Pub. No.: US 2021/0308612 A1
(43) Pub. Date: Oct. 7, 2021

(54) DECONTAMINATION OF OBJECTS

(71) Applicant: Christopher KATO, Edmonton (CA)

(72) Inventor: Christopher KATO, Edmonton (CA)

(21) Appl. No.: 17/223,969

(22) Filed: Apr. 6, 2021

Related U.S. Application Data
(60) Provisional application No. 63/005,579, filed on Apr. 6, 2020.

(51) Int. Cl. B01D 46/00; A61L 2/20; A61L 2/24; A61L 2/26; F24F 8/10

(52) U.S. Cl. CPC B01D 46/0028; B01D 46/006; B01D 46/002; A61L 2/202; A61L 2/24; A61L 2/26; A61L 2202/122; B01D 2279/50; A61L 2202/26; A61L 2202/16; A61L 2202/15; A61L 2202/14; A61L 2202/121; F24F 8/10

(57) ABSTRACT

A system for treating air filters includes a container comprising an air inlet and an air outlet, the container housing an air filter disposed between the air inlet and the air outlet, the air filter being designed to filter airborne pathogens, a vacuum pump connected to a vacuum outlet downstream of the air filter, an ozone generator connected to an ozone inlet upstream of the air filter, and valves that isolate the container from the air inlet and the air outlet and maintain a vacuum applied by the vacuum pump within the container.

### TECHNICAL FIELD

This relates to decontaminating objects of disease-causing pathogens, and in particular, by using vacuum pressure and ozone gas in a decontamination chamber.

### BACKGROUND

In situations where there is a risk of transmission of disease-causing pathogens from an infected host to others, personal protective equipment (“PPE”) may be used to protect the user from becoming infected. PPE may be designed to protect against airborne or droplet infection, which may result from a person coughing, sneezing, or breathing, or direct or indirect physical contact with a contaminated source. Examples of PPE may include clothing such as gowns; gloves; eye protection such as goggles and face shields; face mask or respirators, Some PPE is designed for a single-use, and is discarded after becoming contaminated.

While PPE is critical for healthcare worker who are known to come into contact with individuals who are infected with a pathogen, others that come into contact with large numbers of people or those that come into contact with effluent from humans are also at risk. During widespread infection, PPE may become scarce, and while intended for single use, it may be desirable to reuse the available PPE where possible to conserve resources.

In other situations, air filters may be used to remove dirt, dust, and pathogens from an enclosed space. These filters must be serviced regularly to ensure effectiveness.

### SUMMARY

According to an aspect, there is provided an apparatus for treating contaminated objects. The apparatus comprises an airtight container adapted to contain the objects to be treated, a source of ozone connected to the airtight container, and an outlet from the airtight container. The source of ozone may supply ozone to a bottom zone of the airtight container, and the outlet may be at or toward the top of the airtight container. A vacuum pump is connected to the output.

According to other aspects, the apparatus may comprise the following features, alone or in combination: there may be one or more supports for supporting the objects within the airtight container; the one or more supports may define an equipment receiving area; the inlet may be below the equipment receiving area; the objects to be treated may be personal protective equipment; and the personal protective equipment comprises respirator masks.

According to an aspect, there is provided a system for treating air filters, comprising a container comprising an air inlet and an air outlet, the container housing an air filter disposed between the air inlet and the air outlet, the air filter being designed to filter airborne pathogens; a vacuum pump connected to a vacuum outlet downstream of the air filter; an ozone generator connected to an ozone inlet upstream of the air filter; and valves that isolate the container from the air inlet and the air outlet and maintain a vacuum applied by the vacuum pump within the container.

According to other aspects, the container may comprise a first container and a second container connected in parallel between the air inlet and the air outlet; the valves may isolate each container separately from the air inlet and the air outlet; the system may further comprise a housing that houses the container, the vacuum pump, and the ozone generator; the housing may be portable, the air inlet may comprise flexible ducting that extends from the housing, the air outlet comprises a vent in the housing, and the system may further comprise a blower that moves air between the air inlet and the air outlet; the housing may be installed in the ducting of an HVAC system; the system may comprise a controller that may comprise steps to decontaminate a first filters by: with air flowing from the air inlet to the air outlet through the second filter, operating the valves to isolate the first container from the air inlet and the air outlet and connect the first container to the ozone generator and the vacuum pump; and operating the vacuum pump to apply a vacuum within the first container and introducing ozone from the ozone generator into the first container to reduce the concentration of airborne pathogens on the first filter; the controller may further comprise steps to maintain a vacuum pressure until a predetermined moisture level is achieved in the first container prior to introducing ozone into the first container; the ozone may be introduced into the first container after reducing the vacuum pressure in the first container.

According to an aspect, there is provided a method of removing airborne pathogens from an air mass using a system comprising a first and second filters connected in parallel between an air inlet and an air outlet, the first and second filters being housed in first and second containers respectively, the system comprising valves that separately isolate the first and second containers and connect the first and second containers to an ozone generator and a vacuum pump, the method comprising the steps of: with air flowing from the air inlet to the air outlet through the second filter, decontaminating the first filter by: operating the valves to isolate the first container from the air inlet and the air outlet and connect the first container to the ozone generator and the vacuum pump; and operating the vacuum pump to apply a vacuum within the first container and introducing ozone from the ozone generator into the first container to reduce the concentration of airborne pathogens on the first filter; operating the valves to cause air to flow from the air inlet to the air outlet through the second air filter, and decontaminating the second filter by: operating the valves to isolate the second container from the air inlet and the air outlet and connect the second container to the ozone generator and the vacuum pump; and operating the vacuum pump to apply a vacuum within the second container and introducing ozone from the ozone generator into the second container to reduce the concentration of airborne pathogens on the first filter.

According to other aspects, the container, the vacuum pump, and the ozone generator may be housed in a housing; the housing may be portable and the air inlet may comprise flexible ducting that extends from the housing, the air outlet may comprise a vent in the housing, and the system may further comprise a blower that moves air between the air inlet and the air outlet; the method may further comprise the step of repositioning the flexible ducting; the housing may be installed in the ducting of an HVAC system; the vacuum pressure may be maintained until a predetermined moisture level is achieved in the first container prior to introducing ozone into the first container; the ozone may be introduced into the first container after reducing the vacuum pressure in the first container.

In other aspects, the features described above may be combined in any reasonable combination as will be recognized by those skilled in the art.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is a schematic view of a container used to decontaminate objects.

FIG. 2 is a schematic view of a container housing a filter in a ventilation system.

FIG. 3 is a schematic view of an alternative container housing a filter in a ventilation system.

FIG. 4 is a side elevation view of a portable air decontamination unit.

FIG. 5 is a top plan view of a portable decontamination unit.

FIG. 6 is a side elevation view of a double-filter design.

FIG. 7 is a top plan view of a double-filter design.

FIG. 8 is a top plan view of an air duct.

FIG. 9 is a side elevation view of an air duct.

FIG. 10 is a schematic view of an air decontamination unit.

### DETAILED DESCRIPTION OF PREFERRED EMBODIMENTS

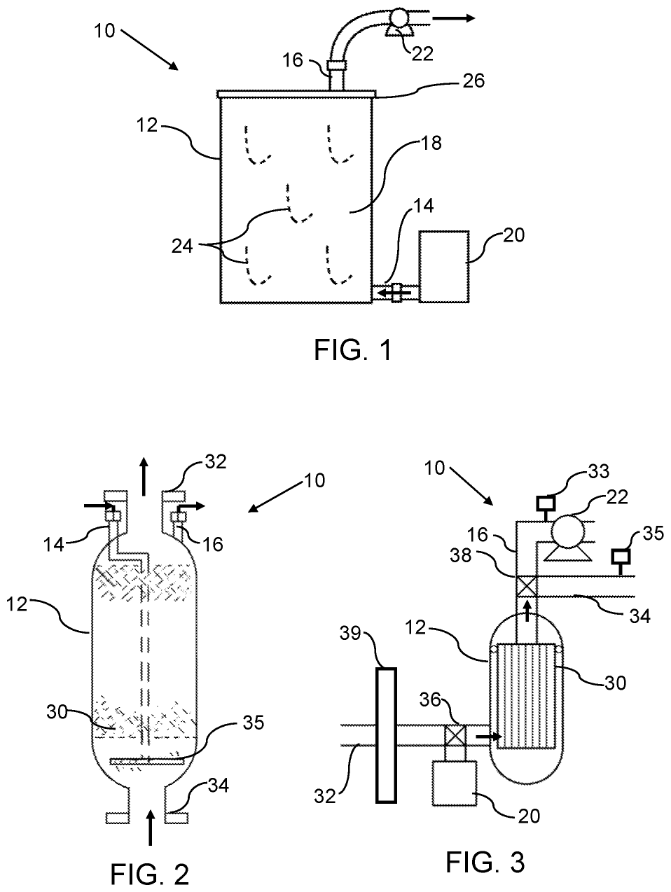

There will now be described an apparatus 10 used to decontaminate objects with reference to FIG. 1 through 10. Apparatus 10 decontaminates objects by applying a vacuum and treating the objects with ozone. Other components may also be incorporated into apparatus 10 to assist in decontaminating the objects, such as UV light or other cleaning fluids, however these will not be described further.

The term decontamination is used herein to refer to the removal of pathogens from an object. This may involve sanitization, sterilization, high level disinfection, or the like, each of which typically refers to the amount by which the amount of pathogen carried by an object is reduced. A desired level of decontamination may be achieved by varying the amount of treatment time, the concentration of ozone, the vacuum applied, or other factors.

Ozone is known to be highly reactive, and to be capable of killing viruses and bacteria. Those skilled in the art will be familiar with the ozone concentrations and dwell times required to achieve a desired level of decontamination, and the types of pathogens that may be resistant to the effects of ozone, or such details may be discovered through routine testing. As such, the extent of the various uses along with the details of each use will not be described herein. However, it is generally understood that a relatively short period of treatment, such as a matter of minutes, may be achieved at sufficient concentrations of ozone.

Depending on the virus or bacteria being treated, the application of vacuum may rupture or otherwise damage the pathogens. Vacuum may also be used to dry the objects being decontaminated as the reduced pressure lowers the boiling point, which may also remove or damage the pathogens.

Referring to FIG. 1, apparatus 10 includes a container 12, an ozone inlet 14, and a vacuum outlet 16. Container 12 is designed with sufficient structural strength to withstand the level of vacuum pressure that will be applied during use. Container 12 has an object receiving area 18 where objects to be cleaned are located during the treatment process. The term “vacuum pressure”, as used herein, is a measure of the reduction of air in a container, i.e. as air pressure decreases, vacuum pressure increases. The units used herein to express vacuum pressure are inches of mercury (inHg).

Ozone inlet 14 may be located toward the bottom of container 12, and may be below object receiving area 18, such that ozone is introduced into a lower zone of container 12 below the objects being treated and travels or diffuses upward toward outlet 16. Inlet 14 may be formed into the bottom surface of container 12 or in the sidewall of container 12 and close to the bottom in a manner that inlet 14 does not interfere with the use of container 12, such as the manner in which container 12 rests on a surface. Ozone inlet 14 may be supplied with ozone from an ozone generator 20, which may be a commercially available unit that may be selected based on the rate or concentration of ozone being generated.

A vacuum source 22 is applied to vacuum outlet 16, which may be at the top of container 12. Vacuum source 22 may be a commercially available source, or may be custom-designed, to apply the necessary level of vacuum.

As ozone is heavier than atmospheric air, placing ozone inlet 14 below vacuum outlet 16 reduces the amount of ozone that is removed via vacuum outlet 16 by displacing air upward, which helps increase the concentration of ozone within container 12.

Ozone generator 20 and vacuum source 22 may be designed or selected based on various factors, such as the volume of container 12, the pathogens to be treated, the target treatment time, the target ozone concentration, etc. In one example, this determination may be based on a desired time period and ozone concentration.

Apparatus 12 may be initially calibrated using a detector to determine the concentration being achieved within the container, after which the apparatus may be operated to achieve the required results based on the predetermined settings and any necessary safety margins.

The amount of vacuum applied may be determined by optimizing the system to ensure sufficient atmospheric air and impurities is removed at a sufficient rate to achieve the desired treatment time and effectiveness.

Optimizations may be achieved through proper testing and routine experimentation. Larger containers may also be used, depending on the type and number of objects to be treated. For example, the size of the container may depend on the type of ozone generator or vacuum pump that is available, or multiple inlets and outlets connected to separate generators and pumps may be used. The size of the container may also be increased if larger objects are being treated. It may be beneficial to introduce the ozone using a manifold, a diffuser, a nozzle, a series of rotating nozzles, etc. in order to encourage a more even distribution of ozone within the container. Other optimizations may be made to improve the treatment technique. The system may also be designed to target specific pathogens or contaminants, if necessary, which may react differently to changes in pressure, moisture levels, etc.

In one example, referring to FIG. 1, apparatus 10 may be designed to treat PPE or other portable objects. The term PPE will be used herein to describe the various objects that may be decontaminated using the apparatus and method described herein, however the discussion herein will be in the context of treating objects used to protect individuals from pathogens, such as face masks and respirators. Other objects that pose a higher risk of transmitting pathogens may also be treated, and preferably objects that are more difficult to clean or decontaminate. Container 12 may have supports 24 in object receiving area 18, such as hooks, support rods, shelves, or the like, that allow the contaminated surfaces of the PPE to be exposed to the ozone. In some examples, supports 24 may be designed to facilitate the placement and removal of PPE within container 12 while ensuring adequate exposure to ozone. Supports 24 may also be part of a removable and replaceable support (not shown), such as a rack that may be pre-loaded with PPE to facilitate loading objects into container 12. The size and shape of object receiving area 18 may depend on the size of container 12, the type of supports 24, and the size of the PPE intended to be treated. Object receiving area 18 may be defined such that the PPE or other object being treated is maintained a minimal distance above the bottom of the container to allow adequate circulation of ozone below the objects being treated.

In one example used to decontaminate respirator or face masks, container 12 may be, or may be sized similar to, a 5-gallon pail with outlet 16 formed in the removable, airtight lid 26 and ozone inlet 14 provided toward the bottom of container 12, and hooks 24 mounted inside container 12 from which the masks are hung. With lid 26 closed, ozone generator 20 and vacuum source 22 may then be operated to treat the masks until they are sufficiently decontaminated. Ozone generator 20 and vacuum pump 22 may be connected to inlet 14 or outlet 16, respectively, by a hose or may be securely attached to the outside of container 12 or lid 26, respectively. Such a container 12 may be able to withstand a vacuum pressure of about 1-inch mercury. If a higher vacuum pressure is desired, the container may be reinforced, or may be designed using different materials, such as high strength polymers or metals, using known principles.

By using commercially available equipment, the example described above may be assembled quickly and relatively low cost as a portable unit that may be used to treat and reuse PPE, such as masks, for those that are at risk of infection. These may be easily manufactured with widely available products and technology and can be made small and light enough to be mobile and used in various circumstances. In other examples an apparatus may be custom designed and manufactured to higher specifications and for a specific use.

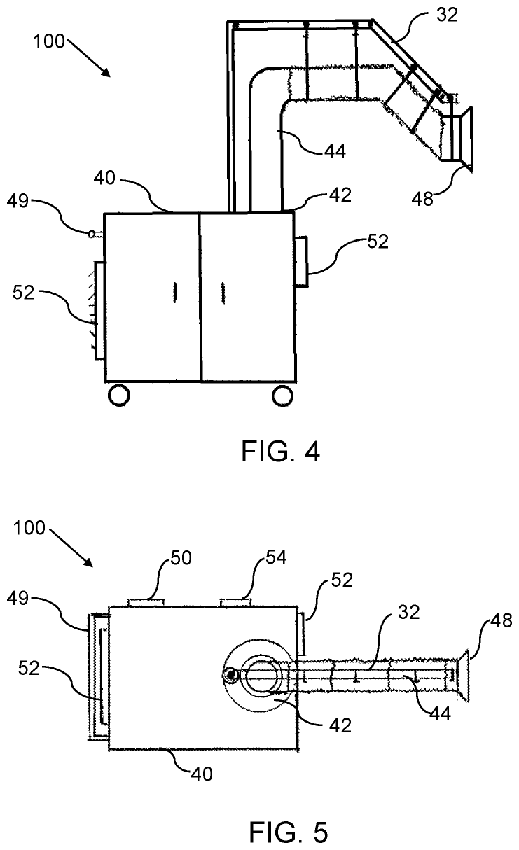

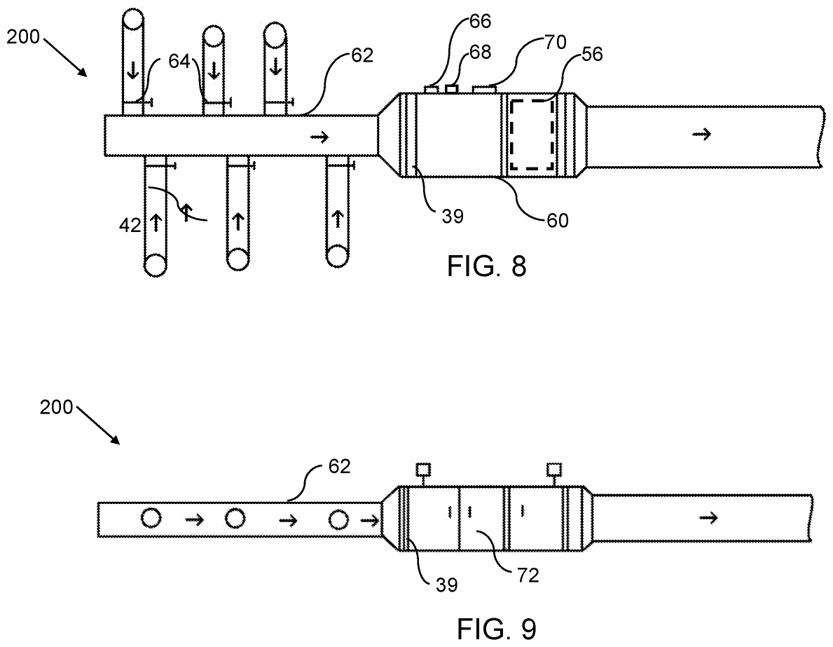

In another example, referring to FIGS. 2 and 3, container 12 may be a housing that houses a filter 30 used to purify air, such as by filtering viruses or other pathogens from an air stream. Two examples will be discussed below. Referring to FIGS. 4 and 5 apparatus 10 may be part of a portable air purifier 100. In another example, referring to FIGS. 8 and 9, apparatus 10 may be part of a fixed air purifier 200, such as may be installed in or as a replacement for an HVAC system. These examples may be used to help decontaminate air in a room, building, or other space, where container 12 is an air filter housing for a filter 30. In other examples, apparatus 10 may be separate from air circulating equipment, in which case filters 30 may be removed from the respective air system and placed in container 12 to be decontaminated before being reinstalled. In other examples, container 12 may be integrated into portable air purifier 10 or HVAC system 200, such that filters 30 may be decontaminated automatically by switching over from air inlets and outlets to ozone generator 20 and vacuum pump 22, as will be described below. It will be understood that other equipment may also be included that treats air, such as a UV light source. However, the discussion below will be given in terms of an air filter as filters typically more likely to need to be decontaminated more regularly, with the understanding that other equipment may also need to be decontaminated, and the discussion below may also be used or adapted to accommodate the other equipment as well using similar principles.

Referring to FIG. 2, container 12 may have an air inlet 32 and air outlet 34 that are separate from ozone inlet 14 and vacuum outlet 16. Inlet and outlet 16 may be located on the same end of container 12 for ease of installation. The flange that defines air outlet 34 may be removable to allow access to filter 30. Ozone inlet 14 may be attached to a nozzle 35 that may be designed to enhance the distribution of ozone within container 12. For example, nozzle 35 may be a manifold, and may rotate as ozone is injected. Nozzle 35 may also be designed with multiple apertures with different orientations to aid in diffusion of ozone within container 12. In this example, filter 30 is treated by closing air inlet 32 and outlet 34, and opening inlet 14 and outlet 16 using valves (not shown) Referring to FIG. 3, air inlet 32 and air outlet 34 are connected to ozone inlet 14 and vacuum outlet 16 by three-way valves 36 and 38, respectively. In addition, as shown in FIG. 3, there may be a secondary filter 39, such as a dust filter used to remove dust upstream of filter 30, such that filter 30 is protected against larger particles, and is therefore more effective for a longer period of time at removing airborne pathogens. There may be sensors 33 and 35 that measure conditions at vacuum outlet 16 and air outlet 34, respectively. Sensors 33 and 35 may measure an airflow rate, vacuum or air pressure, humidity, or other conditions. Sensors 33 and 35 or other sensors may be positioned at any convenient location, such as upstream of container 12, downstream of container 12, or in communication with the interior of container 12 to measure the relevant conditions required to allow a user or a computer controller to make control decisions. These sensors may be incorporated into any example discussed herein, as required.

The examples discussed below will be given in the context of the arrangement shown in FIG. 2, however it will be understood that other arrangements may also be used.

Referring to FIG. 4, an example of a portable air purifier 100 is shown. This unit may be used when it is impractical to modify an HVAC system or when a specific area is of particular concern with respect to the transmission of airborne pathogens. This may include medical or personal care industries such as dentistry, oral surgery, denturists, facial or plastic surgery, etc., where individuals refuse or are unable to wear masks for other reasons, where it is impractical or impossible to wear a protective mask, or where prolonged exposure to others in an enclosed space increases the possibility of transmission. These may include hockey rinks, sport arenas, gymnasiums, workout rooms, classrooms, meeting rooms, etc. As such, portable air purifier 100 provides a more targeted solution that does not include the HVAC environment of other spaces where the risk is less, such as individual offices, large open spaces, and other areas in which protective equipment may be worn and is deemed sufficient.

Referring to FIG. 4, in this example, portable air purifier 100 includes a cabinet 40, such as a rolling cabinet as shown, and a connection 42 connected to a duct 44. Preferably, referring to FIG. 5, connection 42 is a rotating connection and duct 44 is flexible and supported by a support arm 46 to adjust the position of an intake nozzle 48. Purifier 100 may be designed to be more permanent, or semi-permanent, if it is unlikely that it will be moved after installation. Cabinet 40 may be properly counterbalanced or attached to the floor to allow full extension of flexible duct 44 as well as intake nozzle 48 and may include a push handle 49 to make it easier to move cabinet 40 if desired.

Cabinet 40 may be provided with a power connection 50 and a control panel 52 in any convenient location. An exhaust vent 52 may be provided on one wall of cabinet 40 that allows decontaminated or filtered air to return to the working space. An additional connection 54 may be provided that allows another hose or duct to vent purified air to an HVAC air return, bathroom fan, outside, etc. may be provided as well. This may be connected to the vacuum pump, where the amount of removed air will be minimal and may only be required during reclaiming time only. Alternatively, connection 54 or a separate connection may be used to redirect unfiltered air away from the space being protected while filter 30 is being decontaminated.

While not shown, cabinet 40 houses equipment similar to what is shown in FIG. 2 or 3, where air is drawn through filter 30, which is then periodically decontaminated as discussed above.

Air purifier 100 may thus be used to reduce the risk of transmitting airborne pathogens in a particular space or area. By returning the filtered air through exhaust vent 52, no additional demands are placed on the existing HVAC system. In addition, when the filter housed in cabinet 40 is being decontaminated, air may removed from the space and directed to the building HVAC system or the outside via connection 54, such that the space remains protected while minimizing the impact on the existing HVAC system.

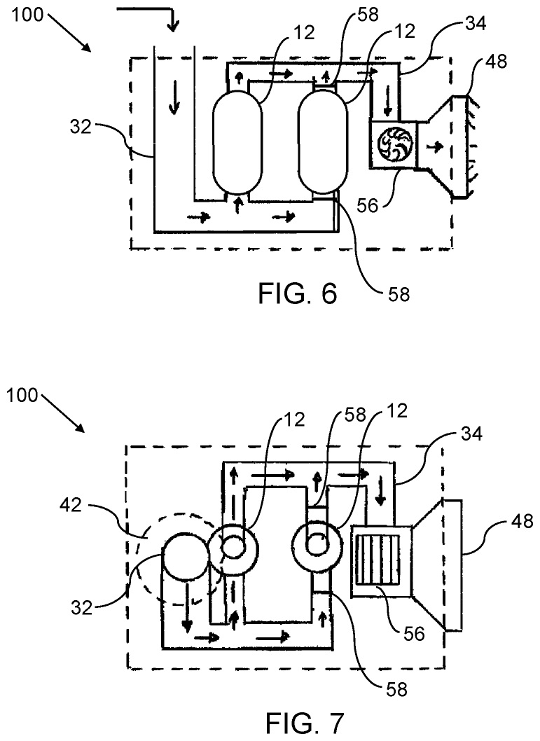

In another example, referring to FIGS. 6 and 7, cabinet 40 may house a double-filter structure, such that filters 30 may be decontaminated while continuing to ensure air is being filtered. One filter 30 may be in a decontamination mode or stand-by mode while the other operates. Alternatively, both may be used simultaneously, with one isolated at a time for decontamination. Valves (not shown) may be used to control the flow of air through filters. Cabinet 40 houses a blower 56 used to draw air through filters 30. As shown, contaminated or untreated air enters inlet connection 42. The air is directed by piping to each filter 30, which are controlled by dampers 58 or valves (not shown) on the inlet and outlet. During a sanitizing cycle or while waiting for the respective filter 30 the dampers are closed not allowing contaminated air in or decontaminated air out. As shown, dampers 58 associated with filter 30 are closed while the other are open. Dampers 58 may be controlled by sensors, time, load, etc.

Referring to FIG. 4, control panel 52 may include features (not shown) such as a status indicator, on/off switch, reclaim indicator lights (cell #1 & cell #2), fan speed adjustment, manual controls, communication equipment for connecting to a network or computer device, etc. Portable air purifier 100 may be controlled by various known strategies, including a processor (not shown) that may be accessed remotely or directly via control panel 52, and may be on a timed schedule and/or may include sensors that initiate a decontamination cycle. In one example, intake nozzle 48 may be stainless steel and include a removable ¾″ mesh screen.

Referring now to FIGS. 8 and 9, an example of a fixed air purifier 200 is shown, designed to be used to help protect an HVAC system against airborne pathogens of an entire building or larger portion of a building treated by a particular HVAC circuit. Fixed air purifier 200 may be suitable for use in bars, casinos, restaurants, library's, halls, etc. and helps support strategies such as social distancing, masking, and addresses moderately hazardous conditions such as where patrons of a restaurant are unmasked but spaced and/or grouped in cohorts, etc. to help protect other occupants. Fixed air purifier 200 may be used to avoid overburdening the existing HVAC system by trying to increase the amount of air exchanges or the amount of replacement air being brought in to address these potential risks.

Referring to FIG. 8, fixed air purifier 200 may include a self-contained cabinet 60 installed in an existing return air duct system 62. Cabinet 60 is preferably installed at a point where air may be conveniently sanitized prior to being ducted to other return ducts and remixed in the central HVAC system. Cabinet 60 may be installed in a ceiling space. Depending on the size of the HVAC system, multiple cabinets 60 may be used at different locations to receive air from different branches of air duct system 62. Air duct system 62 may include dampers 64 as is known in the art.

Cabinet 60 may have a control panel 66 and power connector 68, similar to portable unit 100. However, power connector 68 is more likely to include a hard-wired connection instead of an electrical cord that connects to a wall socket (although either design is possible for each embodiment). Similarly, control panel 66 is more likely to allow for remote control over a computer system or network as access will be more limited. Cabinet 60 may also include a connection 70 for an exhaust hose (not shown) that may be connected to the vacuum pump (not shown). The internal components of cabinet 60 may be similar to those discussed above with respect to cabinet 40 and may include a single filter design, or a double filter design to allow for continuous filtering when one filter is being decontaminated.

Referring to FIG. 9, cabinet 60 may include access doors 72 used to access the components required for the decontamination process discussed above, including a fan blower section, which may be required to achieve proper air distribution due to return air ducting as well as additional air resistance created by the sanitizing cells, etc. The equipment within cabinet 60 may be similar to what is shown in FIGS. 6 and 7 with respect to portable unit 100, with suitable modifications such as the ducting connections, component specifications, etc. A blower may be included or not, depending on characteristics of the existing HVAC system.

Fixed unit 200 may be anchored and supported in a ceiling space using known elements, such as with spring/isolation hangers, and the tie-in for the duct work may include canvas connections.

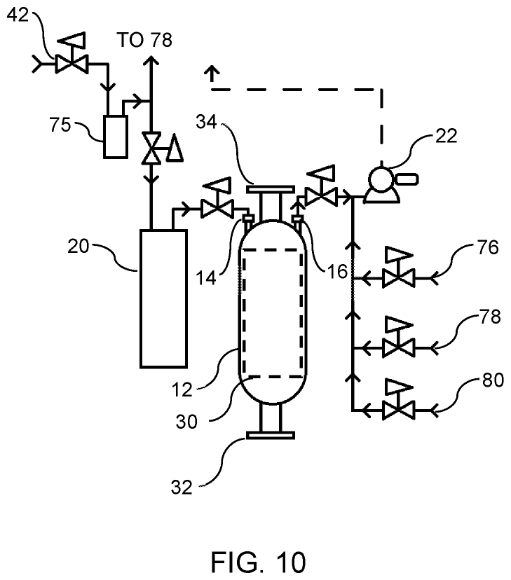

Referring now to FIG. 10, an example of a decontamination cycle is depicted for container 12 as depicted in FIG. 2. With air inlet 32 and air outlet 34 closed, vacuum pump 22 draws down the pressure within container 12 in which filter 30 is positioned. The desired vacuum pressure may be any suitable pressure, with the understanding that a higher pressure is more likely to damage pathogens that are affected by air pressure and is more likely to dry filter 30 (or other equipment). As such, a higher pressure is preferred, and in one example, may be around 27 or 28 inHg. The pressure will depend on various factors, such as the expected humidity in filter 30, the time available to treat filter 30 the type of pathogens expected, the structural strength of container 12 and other components exposed to the vacuum pressure, the turbulence within container 12, etc.

As the pressure is reduced within container 12, the boiling point of water is also reduced, which encourages water to evaporate from filter 30 and is removed from container 12 via vacuum pump 22. It may be desirable to maintain the reduced pressure until the humidity or moisture content of the filter is achieved, which may be measured by a humidity sensor, as this may disrupt or remove some pathogens in filter 30. For example, the vacuum pressure may be maintained until the humidity within container 12 is measured to be around 1-5%. Once the desired humidity level is reached, the vacuum pressure may be reduced, such as to 2 or 3 inHg and ozone may be injected into container 12 using ozone generator 20 and allowed to react with pathogens in filter 30 until sufficiently decontaminated. This time period may be based on measurements or calculations to estimate the rate of decontamination or the amount of ozone required, or it may be based on sensor readings. Once a desired level of decontamination has been achieved, the pressure within container 12 may be returned to normal, and the filter may be returned to service, or held in a stand-by mode. As will be understood, the various dampers and valves may be controlled manually, or by automatic actuators, such as solenoid valves, or other suitable devices. The humidity level within container 12 may be maintained as ozone is injected.

Air from fresh air inlet 74 that is introduced into ozone generator 20 may be pre-conditioned in a conditioning unit 75, which may include a silicon gel filter used to dehydrate the incoming air, a heat source or cooling source to control the temperature, an air-to-air exchanger, etc. Vacuum pump 22 may be connected to an adjacent container 12 (represented by inlet 76) so that the same vacuum pump 22 may be used to treat more than one container, or the conditioning unit 75 via inlet 78 to reclaim the silicon gel, etc., or to a separate inlet 80 that regulates the vacuum.

During normal operation, referring to FIG. 6, the dampers on air inlet 32 and outlet 34 of the appropriate container 12 are opened. Untreated air enters air inlet 32 and is drawn through by an internal blower 56. The untreated air comes in contact with a sanitized filter 30 that traps particulate matter and/or airborne pathogens. This continues until sensors, time load, etc., parameters direct this container 12 filter 30 to close and open the other container that was in reclaim/stand by mode. The recently closed container 12 is then decontaminated as discussed above.

In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be one and only one of the elements.

The scope of the following claims should not be limited by the preferred embodiments set forth in the examples above and in the drawings but should be given the broadest interpretation consistent with the description as a whole.

What is claimed is:

1. A system for treating air filters, comprising:
a container comprising an air inlet and an air outlet, the container housing an air filter disposed between the air inlet and the air outlet, the air filter being designed to filter airborne pathogens;
a vacuum pump connected to a vacuum outlet downstream of the air filter;
an ozone generator connected to an ozone inlet upstream of the air filter; and
valves that isolate the container from the air inlet and the air outlet and maintain a vacuum applied by the vacuum pump within the container.

2. The system of claim 1, wherein the container comprises a first container and a second container connected in parallel between the air inlet and the air outlet, the valves isolating each container separately from the air inlet and the air outlet.

3. The system of claim 2, further comprising a housing that houses the container, the vacuum pump, and the ozone generator.

4. The system of claim 3, wherein the housing is portable, the air inlet comprises flexible ducting that extends from the housing, the air outlet comprises a vent in the housing, and the system further comprises a blower that moves air between the air inlet and the air outlet.

5. The system of claim 3, wherein the housing is installed in ducting of an HVAC system.

6. The system of claim 2, comprising a controller that comprises instructions to decontaminate a first filters by:
with air flowing from the air inlet to the air outlet through the second filter, operating the valves to isolate the first container from the air inlet and the air outlet and connect the first container to the ozone generator and the vacuum pump; and
operating the vacuum pump to apply a vacuum within the first container and introducing ozone from the ozone generator into the first container to reduce a concentration of airborne pathogens on the first filter.

7. The system of claim 6, wherein the controller further comprises instructions to maintain a first vacuum pressure until a predetermined moisture level is achieved in the first container and then to introducing ozone into the first container at a second vacuum pressure that is less than the first vacuum pressure.

8. The system of claim 7, wherein the ozone is introduced into the first container after reducing the vacuum pressure in the first container.

9. A method of removing airborne pathogens from an air mass using a system comprising a first and second filters connected in parallel between an air inlet and an air outlet, the first and second filters being housed in first and second containers respectively, the system comprising valves that separately isolate the first and second containers and connect the first and second containers to an ozone generator and a vacuum pump, the method comprising the steps of:
with air flowing from the air inlet to the air outlet through the second filter, decontaminating the first filter by:
operating the valves to isolate the first container from the air inlet and the air outlet and connect the first container to the ozone generator and the vacuum pump; and
operating the vacuum pump to apply a vacuum pressure within the first container and introducing ozone from the ozone generator into the first container to reduce a concentration of airborne pathogens on the first filter;
operating the valves to cause air to flow from the air inlet to the air outlet through the second filter, and decontaminating the second filter by:
operating the valves to isolate the second container from the air inlet and the air outlet and connect the second container to the ozone generator and the vacuum pump; and
operating the vacuum pump to apply a vacuum pressure within the second container and introducing ozone from the ozone generator into the second container to reduce the concentration of airborne pathogens on the second filter.

10. The method of claim 9, wherein the container, the vacuum pump, and the ozone generator are housed in a housing.

11. The method of claim 10, wherein the housing is portable, the air inlet comprises flexible ducting that extends from the housing, the air outlet comprises a vent in the housing, and the system further comprises a blower that moves air between the air inlet and the air outlet, and further comprising the step of repositioning the flexible ducting.

12. The method of claim 10, wherein the housing is installed in ducting of an HVAC system.

13. The method of claim 9, wherein the vacuum pressure is maintained until a predetermined moisture level is achieved in the first container prior to introducing ozone into the first container.

14. The method of claim 13, wherein the ozone is introduced into the first container after reducing the vacuum pressure in the first container.

15. An apparatus for treating contaminated objects, comprising:
a container sized to contain an object to be treated;
an inlet at or toward a bottom of the container;
an outlet at or toward a top of the container;
an ozone generator connected to the inlet; and
a vacuum generator connected to the outlet.

16. The apparatus of claim 15, comprising one or more supports for supporting the objects within the container.

17. The apparatus of claim 15, wherein the container comprises an equipment receiving area, and the inlet is below the equipment receiving area.

18. The apparatus of claim 15, wherein the object to be treated is personal protective equipment.
